(19) United States
(12) Patent Application Publication
(10) Pub. No.: US 2013/0116314 A1
(43) Pub. Date: May 9, 2013

(54) METHOD OF MAINTAINING OR IMPROVING THE SKIN HEALTH OF AN ANIMAL COMPRISING ADMINISTERING AS A DIETARY SUPPLEMENT A COMPOSITION COMPRISING FLAX SEED OIL AND GAMMA LINOLENIC ACID

(71) Applicant: Lintbells Ltd., Weston Hertfordshire (GB)

(72) Inventors: John Allan HOWIE, Hertfordshire (GB); Robert John Davies, Hertfordshire (GB)

(73) Assignee: Lintbells Ltd., Weston Heartfordshire (GB)

(21) Appl. No.: 13/662,921

(22) Filed: Oct. 29, 2012

Related U.S. Application Data
(62) Division of application No. 12/527,319, filed on Dec. 14, 2009, now abandoned.

(30) Foreign Application Priority Data
Feb. 16, 2007 (GB) 0703087.7

(51) Int. Cl. A61K 31/201; A61K 31/355

(52) U.S. Cl. CPC A61K 31/201; A61K 31/355
USPC 514/458; 514/560

(57) ABSTRACT

A phytochemical composition for use as a veterinary dietary supplement comprising alpha linolenic acid (ALA) and up to 10% gamma linolenic acid (GLA). Preferably the ALA source is flax seed oil and GLA is sourced from borage oil.

### FIELD OF THE DISCLOSURE

The present invention relates to a veterinary phytochemical composition for use as a dietary supplement. The composition has a high omega 3 fatty acid component and further includes an omega 6 fatty acid. Specifically, the composition comprises alpha linolenic acid (ALA) and up to 10% gamma linolenic acid (GLA).

Essential fatty acids (EFAs) are polyunsaturated fatty acids and are the parent compounds of the omega-6 and omega-3 fatty acid series. They are essential in the mammalian diet because there is no synthetic mechanism for them within the body. Humans, for example, can easily make saturated fatty acids or monounsaturated fatty acids with a double bond at the omega-9 position, but do not have the enzymes necessary to introduce a double bond at the omega-3 or omega-6 position.

The omega 6 fatty acid, linoleic acid (LA) and omega 3 fatty acid, alpha-linolenic acid (ALA), are widely distributed in plant and seed oils while fish oils contain the longer-chain omega-3 fatty acids eicosapentenoic acid (EPA) and docosahexenoic acid (DHA). Other marine oils, such as from seal, also contain significant amounts of docosapentenoic acid (DPA), which is also an omega-3 fatty acid. Although the body can, to some extent, convert ALA into these longer-chain omega-3 fatty acids, the omega-3 fatty acids found in marine oils help fulfil the requirement of essential fatty acids.

The fatty acids identified above cannot be made in the body from other substrates and must be supplied in food. Hence, they are called essential fatty acids. In the body, essential fatty acids are primarily used to produce hormone-like substances that regulate a wide range of functions, including blood pressure, blood clotting, blood lipid levels, the immune response, and the inflammation response to injury infection. They also play a key role in skin and coat health in animals such as dogs and cats.

Some of these fatty acids, such as ALA, are sensitive to heat and light whilst others, such as Gamma Linolenic Acid (GLA), are expensive. Therefore, they are rarely included in standard animal diet (complete diets) as the manufacturing processes and cost restrictions make them difficult to include in a product. The degradation of these EFAs in conventional foods leads to rancidity and reduced palatability. Therefore, the standard animal diet is often lacking in these essential fatty acids, potentially having negative effects for the skin and coat.

Research into the need for improving the EFA status of dog diets, particularly in relation to skin and coat health, has been conducted by the Applicant via two surveys, one with vets and the other with dog groomers. In both instances, the professionals identified that at least 40% of dogs that they come into contact with have skin and coat problems and could benefit from a dietary intervention targeted at improved skin and coat health. Accordingly, there is a need for a dietary supplement targeted to improving skin and coat quality.

The current marketed pet care products for skin and coat condition are predominantly curative products i.e. they are designed to treat negative skin and coat conditions already present, and are predominantly targeted at reducing skin irritation and inflammation. In addition, a large proportion of the products are rich in fish oil based omega 3 fatty acids (EPA and DHA), sometimes combined with an omega 6 fatty acid source such as GLA, and general vegetable oils (sunflower or oilseed rape oil) which are high in LA.

The products also contain relatively low or none of the omega 3 EFA, ALA. It should be noted that in the wild, dogs could obtain ALA from eating grass but, for many modern dogs, this is not a readily available source of ALA.

Rees et al, 2001 (Rees, C A; Bauer J E; Burkholder, W J; Kennis R A; Dunbar B L; Bigley K E; Effects of dietary flax seed and sunflower seed supplementation on normal canine serum polyunsaturated fatty acids and skin and hair coat condition scores. Veterinary Dermatology 2001; 12:111-117) conducted a double blind study on 18 dogs, supplementing the dog's feed with either flax seed (high in omega 3 fatty acids) or sunflower seed (high in omega 6 fatty acids). The authors noted a numerical increase in coat scores for both sunflower and flax, although this improvement was not sustained beyond 28 days. An improvement in skin score was only noted for flax seed. It was also noted that the serum phospholipids concentrations of ALA (18:3 n3) were increased with flax seed, but not sunflower seed. In addition, the serum phospholipids concentration of LA (18:2 n6) increased faster in flax seed than in sunflower seed.

LA is well known for its role in skin health and it is possible that the increased accumulation of LA with flax seed consumption could play a role in skin health in this work. The authors speculate that the high level of ALA in the flax seed supplement (˜55%) may compete with LA for delta 6 desaturase (which converts LA to other polyunsaturated fatty acids (PUFAs) in the omega 6 fatty acid chain). This means that, although more LA is available in sunflower seed (LA=75% in sunflower seed and 15% in flax), with sunflower oil consumption, LA is converted to other compounds that play less of a structural role in the skin. On the other hand, more LA is available to the skin when the animal is fed with flax seed as the conversion to other omega 6 fatty acids is slowed.

In a separate paper from the same group (Bauer et al, 1998 (Bauer, J E; Dunbar, B L; Bigley, K E; Dietary flax seed in dogs results in differential transport and metabolism of (n-3) polyunsaturated fatty acids; American Society for Nutritional Sciences. J. Nutr. 1998; 128: 2641S-2644S), the authors fed flax seed and sunflower seed to 18 dogs and measured fatty acid profiles at various time points. For the flax group, the phospholipid fraction was significantly increased for ALA, as might be expected, but also for EPA. EPA is derived from ALA and plays an important anti-inflammatory role in the body. It competes with arachidonic acid (AA) for the enzyme 5-lipoxygenase and thus reduces the production of pro-inflammatory AA metabolites. The same paper also reported a reduction in the level of AA itself, which could be beneficial for reducing inflammation.

Having realised the potential role of omega 3 fatty acids in nutrition for coat and skin health, the Applicant identified a need to develop an improved dietary supplement based on a high content of omega 3 fatty acids that could be proactively used as a maintenance supplement for skin and coat health. Rather than adjusting the core nutritional profile for the animal, the aim of the composition should be to provide suitable nutrition to the animal to ensure that the overall EFA intake of the animal is beneficial to the animal's coat and skin, rather than trying to correct a skin problem after the problem as already occurred.

It was concluded from the work cited above that a supplement based on flax oil, containing both ALA and LA, could beneficial for coat & skin condition in dogs. A selection of a source relatively high level in ALA (>55%) is required to achieve this effect to facilitate faster accumulation of LA. This is important since LA is a structural component of the skin and plays a key role in maintaining the skin's moisture barrier. An increase in the levels of EPA is also important since EPA can act as an anti-inflammatory agent in the skin. In addition, it is desirable to reduce levels of AA which is a pro-inflammatory fatty acid in the omega 6 pathway. A further finding from these papers is that the improved skin and coat scores with flax oil alone were transitory (28 days). Therefore, further development is required to achieve a longer length of benefit, particularly for the skin.

When taking the teachings of Rees et al and Bauer et al into consideration, a further issue is finding an omega 3 source that provides sufficiently high levels of ALA. In addition, it is clear from the teachings of these two papers that a composition comprising omega 3 or omega 6 alone, even when present in a composition at a high level, it not sufficient to provide the desired improvement in coat and skin quality.

The omega 6 fatty acid, Gamma Linolenic Acid (GLA), is known to play a role in skin health, particularly as an anti-inflammatory. In the body, GLA is metabolised from LA using the delta-6-desaturase enzyme. However, as previously noted, high concentrations of ALA can compete for this enzyme activity with LA, which may reduce the availability of GLA to the skin.

Whilst GLA (in the form of Evening Primrose Oil) has been shown to improve skin condition in dogs (Scarff & Lloyd, 1992 (Scarff, D H; Lloyd D H; Double blind, placebo controlled, crossover study of evening primrose oil in the treatment of canine atopy. Vet. Rec. 1992; 131:97-9), it is usually co-formulated with marine fish oil to achieve the anti-inflammatory effects of EPA and DHA. GLA, combined with marine fish oil (EPA and DHA) is used in several current marketed compositions that are sold as curative products (Coatex™, Viacutan™, Efapet™) for skin problems in dogs.

In addition, the main commercial compositions (GLA+fish oil) are based on a curative, anti-inflammatory approach to skin health i.e. fixing a problem that is already present. The approach behind the present invention is to adjust the dietary intake of EFAs such that there is less of a build up of pro-inflammatory compounds such as AA in the first place and to ensure key essential building blocks of the skin, such as LA and GLA, are available. This is a health maintenance approach, rather than a curative approach, although it will manage both situations.

Table 1 sets out an analysis of products currently available on the UK market:

| | | Vitapet | Vetzyme | | | | | | Supadog |
|---|---|---|---|---|---|---|---|---|---|
| | | Moult | Dry | | | | | Omega | essential |
| | Exmarid | Formula | Skin | Coatex | Efavet | Viacutan | Barleans | pet oil | omega oil |
| Level of | Nil | Nil | Nil | Nil | Nil | Nil | High | Med | Nil |
| ALA (Ω3) | | | | | | | (>50%) | (<30%) | |
| Level of | Low | Nil | Medium | High | High | High | Nil | Nil | Medium |
| GLA (Ω6) | | | | | | | | | |

Since the commercial products typically lack any ALA content and so do not provide the benefits previously observed with ALA rich products, it was decided to investigate adding GLA to see whether a synergistic effect could be achieved on skin and coat health over and above the use of flax seed oil alone.

Therefore, the present invention resides in a composition for use as a veterinary dietary supplement comprising omega 3 and omega 6 fatty acids. It has been found that a ratio of omega 6 fatty acid(s) to omega 3 fatty acid(s) of less than 1, preferably from about 0.2 to about 0.9 is ideal

An important factor to be considered when developing veterinary compositions is palatability. Therefore, a composition based on fish oil as a source of omega 3 is to be avoided as animals typically find the taste of these oils unpalatable. As a result, the composition of the present invention is a phytochemical composition comprising omega 3 fatty acids sourced from plants and seeds, rather than fish. The Applicant has found that ALA provides the required omega 3 without the palatability issues of EPA and DHA.

Phytochemicals, sometimes referred to as phytonutrients, occur naturally in vegetables and fruit. They are compounds found in plants and have a beneficial effect on health or an active role in the amelioration of disease.

To provide an improved composition, the Applicant has realised that the benefit period of the composition needs to be extended beyond twenty eight days. This has been achieved through the addition of a source of omega 6 to the composition. Specifically, the omega 6 fatty acid comprises GLA.

Accordingly, the solution to the problem of preparing an improved composition for improving the health and quality of coat and skin in animals has been found to be a phytochemical composition for use as a veterinary dietary supplement comprising ALA, the composition further comprising up to 10% GLA.

The solution to the problem resides in shifting the EFA balance from omega 6 to omega 3, i.e. from LA to ALA. The Applicant has found that if more than 10% GLA is added to the composition, the source of GLA must be increased in the composition. Since any source of GLA also includes LA, an increased amount of GLA increases the LA ratio and reduces the ratio of ALA in the composition. If the ALA ratio drops, the competitive impact on delta 6 desaturase is not achieved, so LA conversion out-competes ALA conversion. In turn, this leads to higher production of AA and less EPA, so the inflammatory balance swings back to pro-inflammatory.

Advantageously, ALA is present in the composition in an amount greater than 55%, preferably in an amount between 40% and 65%. In a preferred embodiment, the composition further comprises 14-18% LA.

Of the major available seeds that contain ALA (flax, hemp, oilseed rape, soya), only flax seed contains sufficient ALA relative to LA to provide over 55% ALA in the base composition. Therefore, a preferred source of ALA is flax seed oil. Ideally the flax seed oil is golden flax seed oil as this variety has a particularly high ALA content (58-60%) and improved palatability (golden flax seed oil is less bitter than brown flax seed oil).

Sources of GLA are Evening Primrose Oil, Borage oil and Blackcurrant seed oil. Preferably the source of GLA in the composition is Borage oil since Borage oil allows the inclusion of higher levels of GLA without dramatically affecting the ratios of ALA and LA in the product.

In a preferred embodiment, the composition of the invention comprises 55% ALA and 1% GLA. To achieve the desired levels of ALA, the minimum ingredient level that flax seed oil can be as part of the composition mix is 95%. If the remainder of the composition is borage oil (5%), this provides a GLA content of 1%. Alternatively, to achieve the minimum desired levels of ALA in a composition, the composition may consist of a minimum ingredient level of 86% flax seed oil and 14% borage oil. This provides a composition comprising about 43% ALA and approximately 3% GLA. Evening Primrose Oil may be used as an alternative to borage oil.

In a yet further preferred embodiment, the composition further comprises about 14-23% LA.

Depending on the sources of ALA and GLA, the composition may also comprise linoleic acid (omega 6) and oleic acid (omega 9) as well as short chain fatty acids.

Advantageously, the composition further comprises Vitamin E to help extend the period of benefit gained from the composition and to act as an anti-oxidant. EFAs, particularly ALA, are susceptible to oxidative attack by free radicals, reducing both their availability and functionality in the body. Vitamin E is believed to play a role in maintaining EFA integrity both in the composition, maintaining availability in the body and maintaining functionality of the EFAs within the skin's metabolism.

Preferably the Vitamin E is provided by a naturally occurring Vitamin E source, such as d-alpha tocopherol

The National Research Council recommends the addition of 1.6 mg Vitamin E per gram of Polyunsaturated Fatty Acid (PUFA) to counter the increased oxidative risk associated with addition of PUFAs to the diet. Therefore, the preferred level of Vitamin E in the composition is between 0.2% and 0.05%. Ideally, Vitamin E is provided by the addition of d-alpha tocopherol in an amount of between 1670 mg/kg d-alpha tocopherol (at manufacture) and 480 mg/kg d-alpha tocopherol (at best before date. There are many possible sources of Vitamin, in particular synthetic tocopherol acetates which are more shelf stable but have less biological activity. Natural d-alpha tocopherol is preferred, to maximise bioavailability and activity or the composition.

In another aspect, the present invention lies in the use of a composition of the invention as a dietary supplement for the maintenance of skin and coat health on an animal selected from the list comprising cat, cow, dog, gerbil, guinea pig, hamster, horse, mouse, rabbit and rat.

The present invention will now be further described by way of a non-limiting example, in which:

### BRIEF DESCRIPTION OF THE FIGURES

FIG. 1 is an illustration of a Visual Analogue Scale question for general skin condition;

FIG. 2 shows a statistically significant improvement in general coat condition at both day 21 and day 42, with a directional move from ‘Dull, dry & coarse’ to ‘Shiny & soft’;

FIG. 3 shows a statistically significant improvement in general skin condition at both day 21 and day 42, with a directional move from ‘Dry & flaky’ to ‘Moist & supple’;

FIG. 4 shows a statistically significant improvement in coat shininess at both day 21 and day 42, with a directional move from ‘Very dull’ to ‘Very shiny’;

FIG. 5 shows a statistically significant improvement in hair loss at both day 21 and day 42, with a directional move from ‘Loses lots of hair’ to ‘Hardly loses any hair’. N.B Higher score=less hair loss;

FIG. 6 shows sub-analysis of the bottom quartile of dogs for pruritis (as measured by scratching) illustrating a dramatic reduction in scratching. (NB High score=less scratching).

FIG. 7 shows sub-analysis of the bottom quartile of dogs for scurfiness (described as dandruff) illustrating a dramatic reduction in scurfiness. (NB−High score=less scurfiness);

FIG. 8 shows a statistically significant improvement in activeness at day 42, but not at day 21, with a directional move from ‘Lethargic & bored’ to ‘Keen to play & active’; and

FIG. 9 shows sub-analysis of the bottom quartile of dogs for improvement in eye shininess illustrating a dramatic increase in eye shininess in these animals.

To prove functionality of the composition, a scientific study on 62 dogs was conducted to measure changes in skin and coat condition during a 6 week feeding programme. Skin and coat condition scores were recorded at day 0, day 21 and day 42. Mean scores for skin and coat condition improved at day 21 and at day 42. The continued improvement in skin and coat condition scores at day 42 demonstrates an improvement over the previous observations by Rees et al, 2001, where use of flax oil alone only provided improvement in condition up to 28 days, after which condition stabilised or declined. The continued improvement in skin and coat condition in this study suggests that the inclusion of GLA with flax oil leads to a synergistic effect on skin and coat condition over and above that achievable with flax oil alone.

### METHODOLOGY

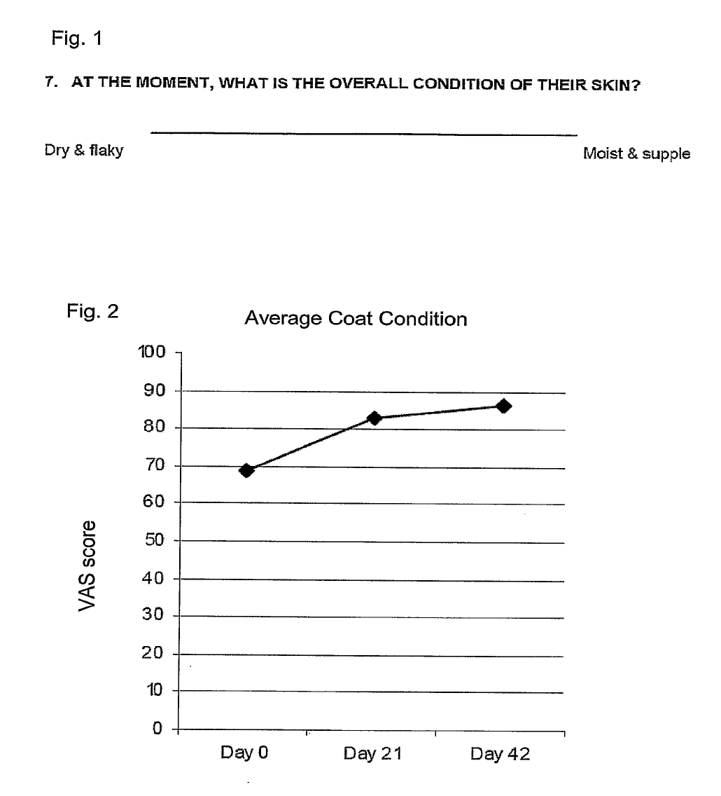

A total of 62 dogs were selected from a consumer research panel. Any dogs already receiving EFA supplements, suffering vet diagnosed diseases, known to be suffering from fleas or pregnant/lactating were excluded from the study. Owners were then asked to complete a Visual Analogue Scale (VAS) form in the presence of the research manager to record the condition of their dog. For each condition component measured, owners were asked to mark the condition of their dog on a 100 mm line, where each end of the line represented the opposite extreme of a condition (see FIG. 1). Owners were asked to place a vertical mark on the line at the place that best represented their dog's condition relative to the two extremes. The form was then collected from the owner.

Owners were then asked to feed their dogs the composition of the invention at the recommended rate for their size of dog (approx 5 ml/10 kg body weight) for a period of six weeks. The composition was made up of 95% golden flax oil and 5% borage oil providing approximately 55% ALA, approximately 15% LA and 1-2% GLA. The composition was added to the dog's main meal.

At 21 days, the owners were visited and asked to complete a second VAS to record the condition of the dog at the 3 week stage. This was also collected from the owner at the time to ensure they had no record of their previous scores. At 42 days, owners completed the final VAS record.

All scores were then converted into a 100 point system and analysed. Seven owners dropped out of the trial during progress, and a further four were excluded from the analysis due to errors in data collection.

During the program a total of seven of the original sixty two dogs discontinued the study. A further four dogs were excluded from the statistical analysis due to incorrect or incomplete recording of VAS record sheets during the study. Analysis was conducted on the Per Protocol (PP) population (51 dogs), with the results shown below. Statistical analysis was conducted using the Wilcoxon Signed Rank test.

### Results

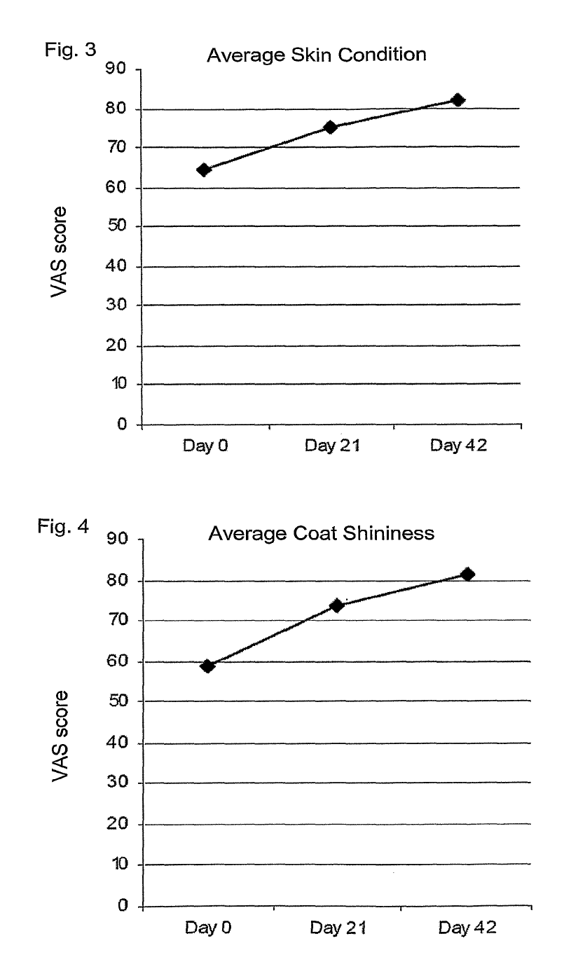

1. Consumption of the composition over a 6 week period resulted in a statistically significant improvement in general coat condition at both day 21 and day 42, with a directional move from ‘Dull, dry & coarse’ to ‘Shiny & soft’ (FIG. 3).

### Wilcoxon Statistical Analysis:

Q10. At the moment, what is the overall condition of their coat?
Shiny and soft ------------------------------------------------ Dull coarse and dry

| | | | |
|---|---|---|---|
| W = | n_{s/r} = | z = | P(1-tail) |

Change from Day 0 to Day 21 delta

| | | | |
|---|---|---|---|
| −738 | 48 | −3.78 | 0.0001 |

Change from Day 21 to Day 42

| | | | |
|---|---|---|---|
| −442 | 51 | −2.07 | 0.0192 |

Change from Day 0 to Day 42

| | | | |
|---|---|---|---|
| −833 | 50 | −4.02 | <0.0001 |

2. Consumption of the composition over a 6 week period resulted in a statistically significant improvement in general skin condition at both day 21 and day 42, with a directional move from ‘Dry & flaky’ to ‘Moist & supple’ (FIG. 4).

### Wilcoxon Statistical Analysis:

Q7. At the moment, what is the overall condition of their skin?
Dry & flaky ----------------------------------------------------- Moist and supple

| | | | |
|---|---|---|---|
| W = | n_{s/r} = | z = | P(1-tail) |

Change from Day 0 to Day 21

| | | | |
|---|---|---|---|
| −559 | 51 | −2.62 | 0.0044 |

Change from Day 21 to Day 42

| | | | |
|---|---|---|---|
| −715 | 51 | −3.35 | 0.0004 |

Change from Day 0 to Day 42

| | | | |
|---|---|---|---|
| −916 | 51 | −4.29 | <0.0001 |

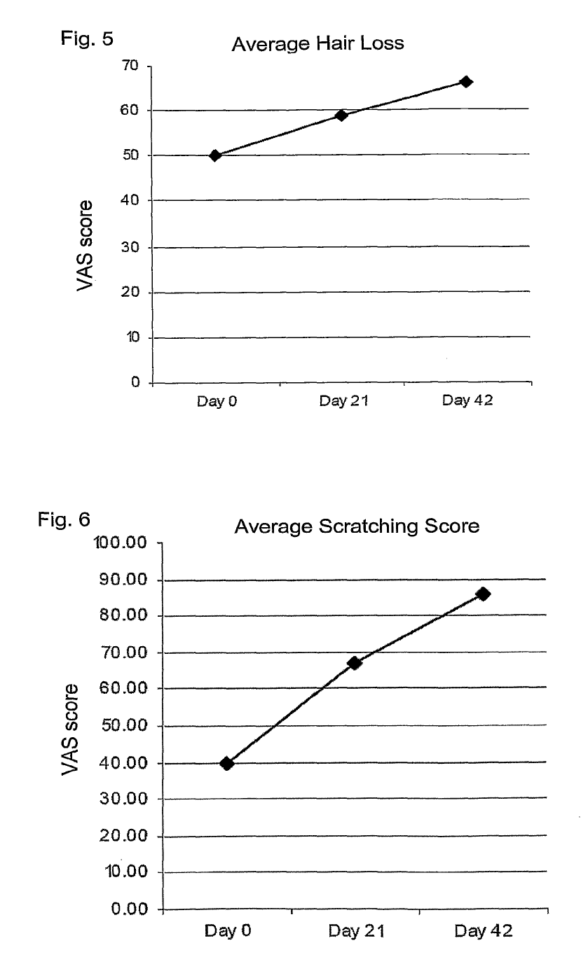

3. Consumption of the composition over a 6 week period resulted in a statistically significant improvement in coat shininess at both day 21 and day 42, with a directional move from ‘Very dull’ to ‘Very shiny’ (FIG. 5).

### Wilcoxon Statistical Analysis:

Q4. At the moment, how shiny is your dog's coat?
Very Dull ---------------------------------------------------------------- Very shiny

| | | | |
|---|---|---|---|
| W = | n_{s/r} = | z = | P(1-tail) |

Change from Day 0 to Day 21

| | | | |
|---|---|---|---|
| −766 | 50 | −3.69 | 0.0001 |

Change from Day 21 to Day 42

| | | | |
|---|---|---|---|
| −708 | 50 | −3.41 | 0.0003 |

Change from Day 0 to Day 42

| | | | |
|---|---|---|---|
| −1050 | 49 | −5.22 | <0.0001 |

4. Consumption of the composition over a 6 week period resulted in a statistically significant improvement in hair loss at both day 21 and day 42, with a directional move from ‘Loses lots of hair’ to ‘Hardly loses any hair’ (FIG. 6). N.B Higher score=less hair loss

### Wilcoxon Statistical Analysis:

Q8. At the moment, does your dog lose much hair?
Hardly loses any hair ------------------------------------------ Loses lots of hair

| | | | |
|---|---|---|---|
| W = | n_{s/r} = | z = | P(1-tail) |

Change from Day 0 to Day 21

| | | | |
|---|---|---|---|
| −506 | 50 | −2.44 | 0.0073 |

Change from Day 21 to Day 42

| | | | |
|---|---|---|---|
| −361 | 49 | −1.79 | 0.0367 |

Change from Day 0 to Day 42

| | | | |
|---|---|---|---|
| −676 | 50 | −3.26 | 0.0006 |

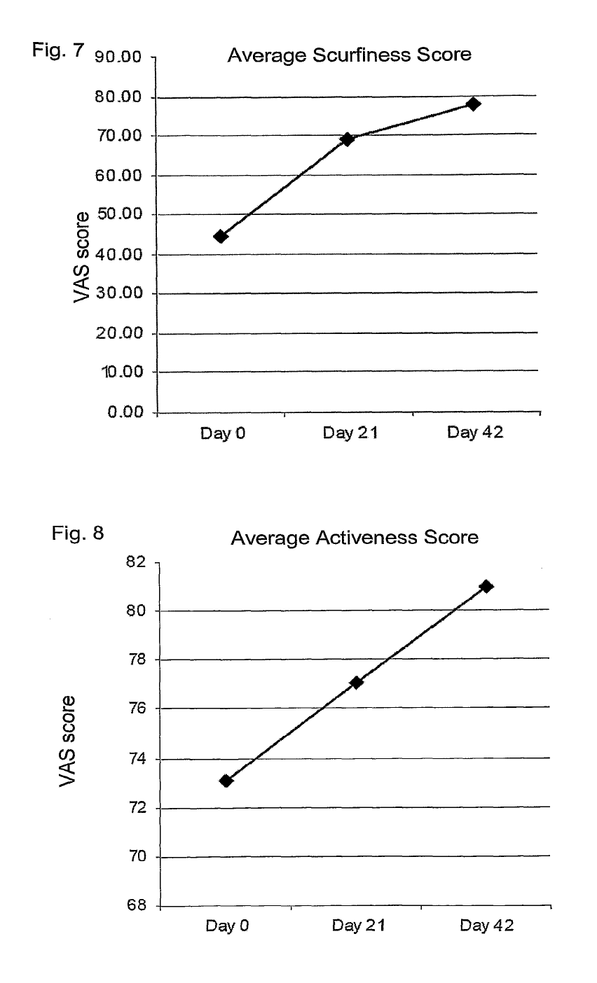

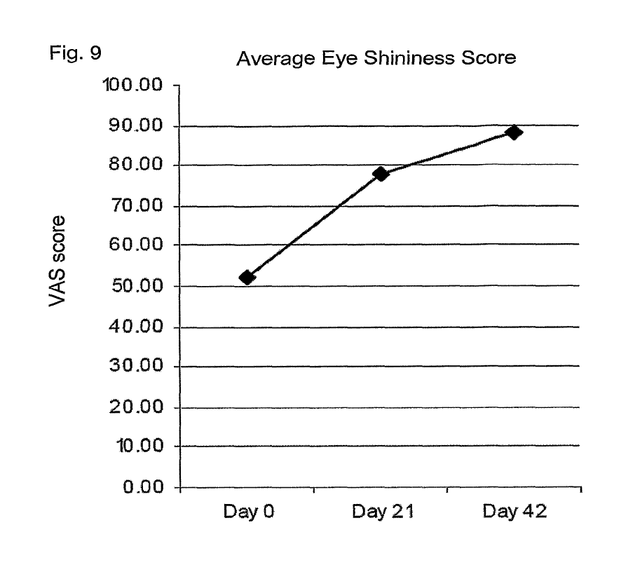

5. Consumption of the composition over a 6 week period resulted in a small but statistically significant reduction in pruritis (as measured by scratching) at day 42, but not at day 21, with a directional move from ‘Scratching all the time’ to ‘Not scratching at all’. FIG. 7 shows sub-analysis of the bottom quartile of dogs for this character showed a dramatic reduction in scratching. (NB−High score=less scratching).

### Wilcoxon Statistical Analysis:

Q5. At the moment does your dog itch or scratch much?
Not scratching at all ------------------------------------- Scratching all the time

| | | | |
|---|---|---|---|
| W = | n_{s/r} = | z = | P(1-tail) |

Change from Day 0 to Day 21

| | | | |
|---|---|---|---|
| −327 | 48 | −1.67 | 0.0475 |

Change from Day 21 to Day 42

| | | | |
|---|---|---|---|
| −296 | 47 | −1.56 | 0.0594 |

Change from Day 0 to Day 42

| | | | |
|---|---|---|---|
| −478 | 50 | −2.3 | 0.0107 |

6. Consumption of the composition over a 6 week period resulted in a small but statistically significant reduction in scurfiness (described as dandruff) at day 42, but not at day 21, with a directional move from ‘Suffers badly from dandruff’ to ‘Doesn't get dandruff at all’. Sub-analysis of the bottom quartile of dogs for this character showed a dramatic reduction in scurfiness. (NB−High score=less scurfiness).

### Wilcoxon Statistical Analysis:

Q9. At the moment, does your dog suffer with any dandruff problems?
Suffers badly from dandruff ----------------------- Doesn't get dandruff at all

| | | | |
|---|---|---|---|
| W = | n_{s/r} = | z = | P(1-tail) |

Change from Day 0 to Day 21

| | | | |
|---|---|---|---|
| −70 | 47 | −0.37 | 0.3557 |

Change from Day 21 to Day 42

| | | | |
|---|---|---|---|
| −622 | 48 | −3.19 | 0.0007 |

Change from Day 0 to Day 42

| | | | |
|---|---|---|---|
| −379 | 47 | −2 | 0.0228 |

7. Consumption of the composition over a 6 week period resulted in a statistically significant improvement in activeness at day 42, but not at day 21, with a directional move from ‘Lethargic & bored’ to ‘Keen to play & active’.

### Wilcoxon Statistical Analysis:

1. Over the last couple of days, how would you describe your dog's
general behaviour?
Lethargic & bored ---------------------------------------- Keen to play & active

| | | | | |
|---|---|---|---|---|
| W = | n_{s/r} = | z = | P(1-tail) | P(2-tail) |

Change from Day 0 to Day 21

| | | | | |
|---|---|---|---|---|
| −295 | 50 | −1.42 | 0.0778 | 0.1556 |

Change from Day 21 to Day 42

| | | | | |
|---|---|---|---|---|
| −198 | 48 | −1.01 | 0.1562 | 0.3125 |

Change from Day 0 to Day 42

| | | | | |
|---|---|---|---|---|
| −694 | 51 | −3.25 | 0.0006 | 0.0012 |

8. Consumption of the composition over a 6 week period resulted in a small but statistically significant improvement in eye shininess at day 42, but not at day 21, with a directional move from ‘Dull & cloudy’ to ‘Bright & shiny’. Sub-analysis of the bottom quartile of dogs for this character also showed a dramatic increase in eye shininess.

### Wilcoxon Statistical Analysis:

Q15. At the moment, how would you describe your dog's eyes?
Dull & Cloudy --------------------------------------------------- Bright and shiny

| | | | |
|---|---|---|---|
| W = | n_{s/r} = | z = | P(1-tail) |

Change from Day 0 to Day 21

| | | | |
|---|---|---|---|
| −219 | 49 | −1.09 | 0.1379 |

Change from Day 21 to Day 42

| | | | |
|---|---|---|---|
| −639 | 46 | −3.49 | 0.0002 |

Change from Day 0 to Day 42

| | | | |
|---|---|---|---|
| −628 | 48 | −3.22 | 0.0006 |

In summary:

a) Consumption of the composition over a 6 week period result in the following statistically significant improvements at both day 21 and day 42:
Improvement in general coat condition
Improvement in general skin condition
Improvement in coat shininess
Reduction in hair loss

b) Consumption of the composition over a 6 week period resulted in the following statistically significant improvements at day 42 only. The average improvement on

1-22. (canceled)

23. A method of maintaining or improving skin health and/or pelage quality of an animal, said method comprising:
administering to an animal as a dietary supplement in an amount effective for maintaining or improving skin health and/or pelage quality of an animal, a phytochemical composition comprising:
(a) a source of Omega-3 fatty acid comprising alpha linolenic acid (ALA); and
(b) a source of Omega-6 fatty acid comprising up to 10% of gamma linolenic acid (GLA),
wherein said composition comprises ALA in an amount ranging from 40-65%.

24. The method of claim 23, wherein the source of Omega-6 fatty acid comprising GLA is chosen from Evening Primrose Oil, Borage oil, Blackcurrant seed oil and combinations thereof.

25. The method of claim 23, wherein the source of Omega-3 fatty acid comprising ALA comprises at least 86% flax seed oil.

26. The method of claim 23, wherein the source of Omega-6 fatty acid comprising GLA is present in the composition in an amount of up to 10%.

27. The method of claim 23, wherein the composition comprises ALA in an amount of at least 55%.

28. The method of claim 27, wherein the composition comprises flax seed oil and borage oil.

29. The method of claim 28, further comprising Evening Primrose Oil.

30. The method of claim 23, wherein the composition consists of 95% flax seed oil and 5% borage oil.

31. The method of claim 23, wherein the composition further comprises linoleic acid (LA).

32. The method of claim 31, wherein the linoleic acid (LA) is present in the composition in an amount ranging from 14 to 23%.

33. The method of claim 23, wherein the composition further comprises Vitamin E in an amount ranging from 0.2% to 0.05%.

34. The method of claim 33, wherein the Vitamin E source is d-alpha tocopherol.

35. The method of claim 34, wherein the d-alpha tocopherol is present in an amount ranging from 1670 mg/kg to 480 mg/kg.

36. The method of claim 23, wherein the administering to the animal comprises administering to an animal chosen from cats, cows, dogs, gerbils, guinea pigs, hamsters, horses, mice, and rabbits.
